(19) United States
(12) Patent Application Publication
(10) Pub. No.: US 2003/0138486 A1
(43) Pub. Date: Jul. 24, 2003

(54) Methods and dosage forms for improving the bioavailability of therapeutic agents

(75) Inventor: Laure Patricia Njiki Ouadji, Laval (CA)

Correspondence Address: Peter J. Manso, Edwards & Angell, LLP, Suite 1150, 350 East las Olas Boulevard, Ft. Lauderdale, FL, 33334, US

(73) Assignee: King Pharmaceuticals Research, Cary, NC

(21) Appl. No.: 10/283,520

(22) Filed: Oct. 29, 2002

Related U.S. Application Data
(60) Provisional application No. 60/346,715, filed on Oct. 29, 2001.

(51) Int. Cl. A61K 9/46; A61K 9/22; A61K 9/14

(52) U.S. Cl. 424/466; 424/468

(57) ABSTRACT

The present invention relates to methods and dosage forms for improving the bioavailability of therapeutic agents that are metabolized in the upper gastrointestinal (GI) tract. The present invention improves the bioavailability of by administering such therapeutic agents in a floating dosage form. The floating dosage forms are kept in the stomach for extended periods of time and the therapeutic agents are not immediately released after ingestion. Controlled release of such therapeutic agents from the dosage forms prevents enzyme saturation thereby improving the bioavailability of such therapeutic agents.

### U.S. PATENT APPLICATION

This application for U.S. patent is filed as a utility application under U.S.C., Title 35, §111(a).

### RELATED U.S. PATENT APPLICATIONS

This application for U.S. patent relates and claims priority to the following U.S. provisional application, which was assigned Serial No. 60/346,715 and filed on Oct. 29, 2001, and is entitled Methods and Dosage Forms for Improving the Bioavailability of Therapeutic Agents, and which is incorporated herein by reference in its entirety.

### FIELD OF THE INVENTION

The present invention relates to methods and dosage forms for improving the bioavailability of therapeutic agents that are metabolized in the upper gastrointestinal (GI) tract. The present invention improves the bioavailability of by administering such therapeutic agents in a floating dosage form. The floating dosage forms are kept in the stomach for extended periods of time and the therapeutic agents are not immediately released after ingestion. Controlled release of such therapeutic agents from the dosage forms prevents enzyme saturation thereby improving the bioavailability of such therapeutic agents.

### BACKGROUND

Many therapeutic agents are metabolized in the upper GI tract into an active Form. This active form is then absorbed through the wall of the intestine. The therapeutic agents are metabolized by enzymes present in the upper GI tract. If the therapeutic agent is present in large quantities, saturation of these enzymes can occur with the result that most of the therapeutic agent passes through the GI tract without being metabolized and therefore limits the potency of the therapeutic agent.

Conventional controlled release dosage forms have a density greater than that of gastric contents, thus these dosage forms sink to the bottom of the stomach once ingested. The de novo design of oral controlled drug delivery systems (DDS) is known in the art to achieve more predictable bioavailability of drugs. However, it is well known that conventional controlled release and sustained release DDS do not Overcome adversities such as gastric residence time (GRT) and gastric emptying time (GET). Gastric emptying is the process by which the fasted stomach exhibits a cyclic activity called the interdigestive migrating motor complex (IMMC). The purpose of this cycle is to migrate the contents of the stomach through the pyloric sphincter although ingestion of food interrupts the cycle. See Moes: Critical Reviews in Therapeutic Drug Carrier Systems, 10(2):143-195 (1993). Due to the IMMC, gastric residence of the stomach contents, including pharmaceutical forms, is short.

One approach to overcome the adversities of GRT and GET is the floating system also known as hydrodynamically balanced systems. These systems are expected to remain lastingly buoyant on the gastric contents without affecting the intrinsic rate of emptying because their bulk density is lower than that of the gastric fluids. See Moes: Critical Reviews in Therapeutic Drug Carrier Systems, 10(2):143-195 (1993). The floating forms maintain their low density value while the polymer hydrates and forms a gel. The drug is progressively released from the swollen matrix as in the case of conventional hydrophilic matrices. Id.

U.S. Pat. No. 4,167,558 to Sheth et al. discloses a sustained release formulation, which is retained in the stomach and which slowly releases acetylsalicylic acid. The sustained release formulation, disclosed by Sheth et al., utilizes a hydrocolloid, which in contact with gastric fluid at body temperature will form a soft Gelatinous mass on the surface of the tablet, thus causing it to enlarge somewhat and acquire a bulk density (specific gravity) of less than one. Since the bulk density of the tablet is less than that of the gastric fluid, the tablet remains floating in the gastric fluid and thereby avoids being eliminated from the stomach during gastric emptying. The acetylsalicylic acid is then slowly released from the gelatinous mass via diffusion.

U.S. Pat. No. 5,169,638 to Dennis et al. discloses a loose powder filled capsule that is buoyant so that it will float on gastric juices and thereby improve drug availability. Dennis et al. teach that the buoyant controlled release powder formulation will release a pharmaceutical of, a basic or alkaline character at a controlled rate relatively independent of the pH of the environment such that in vivo consistent release is achieved throughout the gastrointestinal tract.

U.S. Pat. No. 4, 814, 179 to Bolton et al. discloses a non-compressed tablet that contains the therapeutic agent, gelling agent, a therapeutically acceptable inert oil and water. The tablet has a bulk density of less than one and, therefore, floats on gastric fluid and delivers the therapeutic agent over an extended period of time.

The following is a list of drugs explored for various floating dosage forms. See Singh et al., Journal of Controlled Release, 63:240 (2000), which is incorporated herein by reference in its entirety.

| | |
|---|---|
| Micro- | Aspirin; griseofulvin and p-nitroaniline; ibuprofen; |
| spheres: | terfenadine; tranilast. |
| Granules: | Diclofenac sodium; Indomethacin; Prednisolone. |
| Tablets/Pills: | Acetominophen; acetylsalicylic acid; amoxycillin |
| | trihydrate; ampicillin; atenolol; chlorpheniramine maleate; |
| | cinnarizine; diltiazem; fluorouracil; isosorbide |
| | mononitrate; isosorbide dinitrate; p aminobenzoic acid; |
| | piretanide; prednisolone; quinidine gluconate; riboflavin |
| | 5'-phosphate; sotalol; theophylline; verapamil HCl. |
| Capsules: | Chlordiazepozide; diazepam; furosemide; L-dopa and |
| | bensarazide; misoprostol; propranolol HCI; |
| | Ursodeoxycholic acid.. |
| Films: | Cinnarizine. |

There is no suggestion in any of the prior art that floating dosage form technology does or can be utilized to improve the bioavailability of therapeutic agents that are metabolized in the upper gastrointestinal tract. Consequently, there is a need for controlled release dosage forms that can improve the bioavailability of therapeutic agents that are metabolized to active forms in the upper gastrointestinal tract.

### SUMMARY OF THE INVENTION

The present invention overcomes and alleviates the above-mentioned drawbacks and disadvantages in the drug-delivery art through the discovery of novel formulations suitable for the preparation of controlled release dosage forms for oral administration. The formulation comprises a therapeutic agent that is metabolized in the upper GI tract in combination with a controlled-release agent so as to be hydrodynamically balanced so that, in contact with gastric fluid, they have a bulk density less than one g/ml and therefore are buoyant in the gastric fluid. Such formulation is retained in the stomach during the time when substantially all of the medicaments are released therefrom.

The novel method and composition described herein is an original solution to the problem of enzyme saturation of therapeutic agents that are metabolized in the upper GI tract. Never before was it suggested that the floating tablet delivery system could be utilized to avoid enzyme saturation. Instead, all the prior art reports that the floating delivery system is a solution to short gastric residence time and gastric Emptying time. See Stockwell et al.: Journal of Controlled Release, 3:167 (1986)(Most drugs are optimally absorbed in the small intestine, therefore rapid gastric emptying normally leads to an early biological availability of the drug. In contrast, delayed gastric emptying can provide a prolonged action); Singh et al.: Journal of Controlled Release, 63:235, 237 (2000)(While the system is floating on the gastric contents, the drug is released slowly at a desired rate from the system. This results in an increase in the GRT and a better control of fluctuations in plasma drug concentrations in some cases); and Ingani et al.: International Journal of Pharmaceutics, 35:157-164 (1987) (This study has shown that the bilayer floating dosage form can be opportunely utilized to increase the gastric residence time of a drug to improve its biological availability), each of which is incorporated by reference in their entireties.

Utilizing this method for oral administration of therapeutic agents that are metabolized in the upper GI tract improves their bioavailability. The release of these therapeutic agents over time rather than the normal immediate release, which overloads and saturates the enzymes, a more steady concentration of substrate over time prevents the excess substrate from passing the absorption site and thereby being eliminated from the body.

The advantages of this invention include, but are not limited to: (1) administration of lower doses of therapeutic agents; (2) better control over serum levels of the therapeutic agent; and (3) decrease in non-absorption of the therapeutic agent.

In accordance with the invention, there is provided pharmaceutical controlled release dosage forms, comprising at least one therapeutic agent or pro-drug that is metabolized in the upper GI tract into an active form, at least one controlled-release agent and other excipients such as, for example, porosity agents. These controlled release dosage forms are prepared as a floating drug device, which will remain buoyant on the gastric fluids in the stomach after oral administration. Such pharmaceutical dosage forms will release the medicament over an extended period of time so that delivery of the therapeutic agent to the small intestine will occur steadily rather than immediate release. Such steady release over time of the therapeutic agent at the metabolisation and absorption site will prevent enzyme saturation and thereby exhibit greater bioavailability of the active.

### DETAILED DESCRIPTION OF THE INVENTION

By way of illustrating and providing a more complete appreciation of the present invention and many of the attendant advantages thereof, the following detailed description is given concerning the novel oral methods of treatment and dosage forms for improving the bioavailability of therapeutic agents that are metabolized in the gastrointestinal tract into active forms.

Many therapeutic agents are converted to more active species by upper gastrointestinal enzymes. High release rates of therapeutic agents results in overloading of these upper gastrointestinal enzymes. The methods of treatment and dosage forms of the present invention reduce the rate of dissolution of therapeutic agents while maintaining the dosage form in the stomach for an extended period of time such that the upper gastrointestinal enzymes are not overloaded and the patient receives relatively constant plasma levels of the therapeutic agent over an extended period of time.

The controlled release of therapeutic agents has been the subject of extensive Research over the last half of the twentieth century. Controlled release of therapeutic agents is of high importance because such release allows for once- or twice-a-day dosage regimens, which ease the burden on the patient and therefore lead to increased patient compliance. There exists several types of polymers that have been used as matrices for the controlled release of drugs. Polymeric materials such as Polyvinyl chloride, polyethylene, polyamides, ethylcellulose, silicone, polyhydroxylethyl methacrylate, acrylic co-polymers, polyvinylacetate-polyvinyl chloride co-polymers and other polymers have been described as adequate matrices for controlled release dosage form preparation. See, for example, U.S. Pat. No. 3,087,860, U.S. Pat. No. 2,987,445, and Pharm. Acta Helv., 55:174 (1980), which are incorporated herein by reference in their entireties.

Starch is one of the most attractive biopolymers for use as a controlled release matrix since it can be mass produced with a high purity at a very economical price. Amylose is a natural substance obtained from starch. It is essentially a linear, non branched, polymer of glucopyranose units with a-D-(1-4) linkages. In starch, Amylose is usually accompanied by amylopectin, which is a branched polyglucose polymer with a significant frequency of branching points based on a-(1-6) glucosidic bonds.

Cross-linked starch is a controlled release matrix in solid dosage forms. Cross linked starch is produced by the reaction of starch with a suitable cross-linking agent such as, for example, 2,3-dibromopropanol, epichlorohydrin, phosphorous oxychloride and sodium trimetaphosphate or by thermal cross-linking. A key feature of cross-linked starch is its ability to release therapeutic agents as a constant rate, Following zero order kinetics, such as described in S.T.P. Pharma, 1986, 2, 38. Cross-linked starch maintains this constant rate controlled release because it Functions as a swelling controlled system. Such systems consist of glassy polymers into which a water front penetrates at a constant rate. Behind this front, the polymer is in a rubbery state. Zero order release is obtained when the diffusion coefficient of the therapeutic agent in the rubbery polymer is much higher than in the glassy polymer. Cross-linked starches yielding these desired characteristics are described in U.S. patent application Ser. Nos. 09/028,385, 09/257,090, and 09/606,399, each of which is incorporated herein by reference in their entirrties.

The methods of treatment and dosage forms of the present invention utilize a controlled release agent incorporated into a floating gel dosage form. Any dosage form currently known in the art such as, for example, tablets, caplets, bilayer tablets, bilayer caplets, dry coated tablets, dry coated caplets, film coated tablets, film coated caplets, capsules and film coated capsules may be used as the dosage forms of the present invention. Such floating gel dosage forms are maintained in the stomach of the patient for extended periods of time because they have a relative density less than 1 g/ml and therefore remain buoyant on the gastric fluids of the patient. The floating gel dosage forms may be either of uniform or bi-layer construction.

In the case of uniform construction, the therapeutic agent, controlled release matrix, optional buoyancy agent and any other additives are homogeneously mixed and then formed into the desired dosage form. The buoyancy agent is optional Because many controlled-release agents have a relative density such that a dosage form manufactured from them has a low enough relative density to remain buoyant. In the case of bi-layer construction, the therapeutic agent and controlled release matrix are homogeneously mixed and formed into a dosage form. This dosage form is then combined with a dosage form comprising an optional buoyancy agent and any other additives to yield a bi-layer construction dosage form, wherein the one layer Provides the buoyancy and the other layer provides the controlled release of the therapeutic agent. Preferably, the dosage form comprises a dosage form of uniform construction, as this construction allows for more efficient manufacture of the dosage form.

Various optional buoyancy agents may be used to maintain the relative density at a value of less than 1 g/ml. Such buoyancy agents are celluloses, gums, polysaccharides including starch and starch derivatives and gelatin. Preferred buoyancy agents are hydrocolloids. The most preferred buoyancy agents are the various hydroxypropymethylcelluloses. Various materials may be added to the buoyancy agent to improve its cohesion such as, for example, magnesium stearate or various fatty substances. Optionally, sodium bicarbonate, sodium carbonate, calcium carbonate, lysine carbamate or any other agent that produces carbon dioxide (C02) gas when contacted with gastric acidity or an optional pharmaceutically acceptable acid such as, for example, citric acid or tartaric acid in the matrix can be used to increase buoyancy.

Additionally, any of the well known diluents such as, for example, lactose, sorbitol, mannitol, glucose, microcrystalline cellulose, gelatin, starch, dicalcium phosphate and polyethylene glycol may be added to the dosage form. Preferred diluents are porosity agents, such as, for example, lactose.

Thus, the methods of treatment and dosage forms of the present invention utilize a controlled release matrix in combination with an optional buoyancy agent to improve the bioavailability of a highly soluble therapeutic agent by increasing the dosage form's gastric residence time and providing controlled release of the therapeutic agent from the dosage. The combination of increased gastric residence time and controlled release maintains relatively constant plasma levels by avoiding the overloading of upper gastrointestinal enzymes.

The dosage forms of the present invention may be prepared by any method known to one of ordinary skill in the art. The dosage form may be tablets, coated tablets, capsules or any other form known to one of ordinary skill in the art. Preferably, the dosage form is a gelatin or hydroxypropylmethylcellulose (HPMC) capsule, wherein the therapeutic agent, controlled release agent, optional buoyancy agent and any additional additives or diluents are homogeneously mixed throughout the capsule.

The following examples are given by way of illustration only and are not to be considered limitations of this invention or many apparent variations of which are possible without departing from the spirit or scope thereof.

### EXAMPLE 1

A solid, controlled release, floating gel dosage form was prepared by homogeneously mixing 12 mg of ramipril, 34.4 mg of CONTRAMID®, 34.4 mg of HPMC K4M, 43.6 mg of lactose and 0.6 mg of silicon dioxide. The homogeneously blended mixture was placed inside a HPMC capsule.

### EXAMPLE 2

A solid, controlled release, floating gel dosage form was prepared by homogeneously mixing 12 mg of ramipril, 32.4 mg of CONTRAMID®, 32.4 mg of HPMC K4M and 48.2 mg of mannitol. The homogeneously blended mixture was placed inside a HPMC capsule.

### EXAMPLE 3

A solid, controlled release, floating gel dosage form was prepared by homogeneously mixing 12 mg of ramipril, 34.8 mg of CONTRAMID®, 34.8 mg of HPMC K4M and 43.4 mg of polyethyleneglycol 8000. The homogeneously blended mixture was placed inside a HPMC capsule.

### EXAMPLE 4

A solid, controlled release, floating gel dosage form was prepared by homogeneously mixing 12 mg of ramipril, 33.2 mg of CONTRAMID®, 33.2 mg of HPMC K4M and 46.6 mg of polyethyleneglycol 8000. The homogeneously blended mixture was placed inside a HPMC capsule.

### EXAMPLE 5

A solid, controlled release, floating gel dosage form was prepared by homogeneously mixing 12 mg of ramipril, 30.4 mg of CONTRAMID®, 30.4 mg of HPMC K4M, 47.8 mg of lactose, 3.1 mg of sodium bicarbonate and 1.3 mg of sodium stearyl fumarate. The homogeneously blended mixture was placed inside a HPMC capsule.

### EXAMPLE 6

A solid, controlled release, floating gel dosage form was prepared by homogeneously mixing 12 mg of ramipril, 31.0 mg of CONTRAMID®, 31.0 mg of HPMC K4M, 47.8 mg of lactose, 1.9 mg of sodium bicarbonate and 1.3 mg of sodium stearyl fumarate. The homogeneously blended mixture was placed inside a HPMC capsule.

### EXAMPLE 7

A solid, controlled release, floating gel dosage form was prepared by homogeneously mixing 12 mg of ramipril, 31.7 mg of CONTRAMID®, 31.7 mg of HPMC K4M, 47.7 mg of lactose, 0.6 mg of sodium bicarbonate and 1.3 mg of sodium stearyl fumarate. The homogeneously blended mixture was placed inside a HPMC capsule.

### EXAMPLE 8

A solid, controlled release, floating gel dosage form was prepared by homogeneously mixing 8 mg of ramipril, 31.6 mg of CONTRAMID®, 31.6 mg of HPMC K4M, 49.5 mg of lactose, 3.1 mg of sodium bicarbonate and 1.2 mg of sodium stearyl fumarate. The homogeneously blended mixture was placed inside a HPMC capsule.

### EXAMPLE 9

A solid, controlled release, floating gel dosage form was prepared by homogeneously mixing 8 mg of ramipril, 32.9 mg of CONTRAMID®, 32.9 mg of HPMC K4M, 49.5 mg of lactose, 0.6 mg of sodium bicarbonate and 1.2 mg of sodium stearyl fumarate. The homogeneously blended mixture was placed inside a HPMC capsule.

The capsules prepared in Examples 1-9 were subjected to dissolution assays designed to mimic the gastric fluid of a patient. The capsules of examples 1-9 exhibited controlled release of ramipril. The data for 90% release of ramipril from each capsule is presented in Table 1.

TABLE 1
Time measured in hours for 90% release of therapeutic agent from solid,
controlled release, floating gel dosage forms.

| Example | 1 | 2 | 3 | 4 | 5 | 6 | 7 | 8 | 9 |
|---|---|---|---|---|---|---|---|---|---|
| hours | 5.8 | 5.0 | 2.9 | 2.0 | 2.1 | 2.9 | 5.0 | 2.4 | 3.3 |

The capsules prepared in Examples 5-9 were subjected to buoyancy studies designed to mimic the gastric fluid of a patient. The capsules of Examples 5-9 were placed into a Vankel 7000 test station containing 500 g of dissolution medium consisting of a pH 3.0 buffer of 1.5 ml of 12M HCI in 20.0 L of deionized water. The capacity to float was checked visually at 30 minute intervals. The data for the buoyancy of each capsule is presented in Table 2.

TABLE 2
Time measured in hours for the buoyancy of solid, controlled release,
floating gel dosage forms.

| | | | | | |
|---|---|---|---|---|---|
| Example | 5 | 6 | 7 | 8 | 9 |

| | | | | | |
|---|---|---|---|---|---|
| Hours | 2.1 | 2.9 | 5.0 | 2.4 | 3.3 |

While the present invention has been described in the context of embodiments and examples and are well suited to fulfill the objectives stated above, it will be readily apparent to those skilled in the art that other modifications and variations can be made therein without departing from the spirit or scope of the present invention. Thus, it will be appreciated that numerous modifications and other embodiments may be implemented by those skilled in the art, and it is intended that the appended claims cover all such modifications and embodiments that fall within the true spirit and scope of the present invention. Accordingly, it is not intended that the present invention be limited to the specifics of the foregoing description of the embodiments and examples, but rather as being limited only by the scope of the invention as defined in the claims appended hereto.

Having described my invention, I claim:

1. A solid controlled-release oral pharmaceutical dosage form, comprising:
a therapeutic agent that is metabolized in the upper gastrointestinal tract; and
a controlled release agent,
wherein the dosage form maintains a bulk density of less than about one g/ml, thereby increasing the gastric residence time of the dosage form resulting in an increase in the bioavailability of the therapeutic agent.

2. The solid controlled-release oral pharmaceutical dosage form according to claim 1, further comprising a buoyancy agent.

3. The solid controlled-release oral pharmaceutical dosage form according to claim 1, further comprising a porosity agent.

4. The solid controlled-release oral, pharmaceutical dosage form according to claim 1, wherein the therapeutic agent is an angiotensin converting enzyme inhibitor.

5. The solid, controlled-release oral, pharmaceutical dosage form according to claim 4, wherein the angiotensin converting enzyme inhibitor is ramipril.

6. The solid, controlled-release oral, pharmaceutical dosage form according to claim 2, wherein the buoyancy agent is selected from the group consisting of hydroxypropylmethylcellulose and polyethyleneglycol.

7. The solid, controlled-release oral, pharmaceutical dosage form according to claim 2, wherein the buoyancy agent is selected from the group consisting of sodium bicarbonate, sodium carbonate, calcium carbonate and lysine carbamate and optionally further comprising a pharmaceutically acceptable acid selected from the group consisting of citric acid and tartaric acid.

8. The solid, controlled-release oral, pharmaceutical dosage form according to claim 1, wherein the controlled release agent is a cross-linked high amylose starch, comprising a mixture of from about 10% to about 60% amylopectin and from about 40% to about 90% amylose.

9. The solid, controlled-release oral, pharmaceutical dosage form according to claim 8, wherein the cross-linked high amylose starch has been covalently cross-linked with a covalent cross-linking agent.

10. The solid, controlled-release oral, pharmaceutical dosage form according to claim 9, wherein the covalent cross-linking agent is selected from the group consisting of 2,3-dibromopropanol, epichlorohydrin, phosphorous oxychloride and sodium trimetaphosphate.

11. The solid, controlled-release oral, pharmaceutical dosage form according to claim 3, wherein the porosity agent is selected from the group consisting of lactose, mannitol and sodium bicarbonate.

12. The solid, controlled-release oral, pharmaceutical dosage form according to claim 11, wherein the porosity agent is lactose.
